(19) United States
(12) Patent Application Publication
(10) Pub. No.: US 2017/0022057 A1
(43) Pub. Date: Jan. 26, 2017

(54) PROCESS FOR GENERATING SYNGAS FROM A CO2-RICH HYDROCARBON-CONTAINING FEED GAS

(71) Applicants: Harald Ranke, Pöcking (DE); Marcus Guzmann, Münsing (DE); Rachid Mabrouk, München (DE); Ulrich Heucke, München (DE)

(72) Inventors: Harald Ranke, Pöcking (DE); Marcus Guzmann, Münsing (DE); Rachid Mabrouk, München (DE); Ulrich Heucke, München (DE)

(21) Appl. No.: 15/058,182

(22) Filed: Mar. 2, 2016

(30) Foreign Application Priority Data
Mar. 15, 2015 (DE) DE 102015004214.9

(51) Int. Cl. C01B 3/48; C01B 3/36; C01B 3/56; C10G 2/00; B01D 53/047; C07C 29/151; F01K 7/16; F01K 25/08; E21B 43/16; B01D 53/14; C01B 3/38; C10G 45/00

(52) U.S. Cl. CPC C01B 3/48; C01B 3/384; C01B 3/36; C01B 3/56; C10G 2/30; C10G 45/00; C07C 29/151; F01K 7/16; F01K 25/08; E21B 43/164; B01D 53/1425; B01D 53/1475; B01D 53/1468; B01D 53/1493; B01D 53/047; C01B 2203/0233; C01B 2203/0255; C01B 2203/0283; C01B 2203/042; C01B 2203/0475; C01B 2203/061; C01B 2203/062; C01B 2203/84; C01B 2203/0827; C01B 2203/1258; C01B 2203/1235; C01B 2203/142; C01B 2203/146; B01D 2252/2021; B01D 2259/402; B01D 2259/404; B01D 2257/504; B01D 2259/40056

(57) ABSTRACT

A process for generating a syngas from a CO₂-rich and hydrocarbon-containing feed gas, wherein a CO₂-rich and hydrocarbon-containing feed gas is provided and is reacted in a syngas generation step by means of partial oxidation or steam reforming to give an H₂- and CO-comprising syngas. At least CO₂ is removed from the feed gas in a scrubbing of the feed gas by means of a scrubbing medium, before the feed gas is fed to the syngas generation step.

The invention relates to a process for generating a syngas from a hydrocarbon-containing feed gas.

In this case, a hydrocarbon-containing feed gas that comprises methane is provided and is reacted in a syngas generation step by means of partial oxidation and/or steam reforming to give an H₂- and CO-comprising syngas.

Such processes are known, e.g. from US2012/0326090 A1, US2012/0282145 A1, US2013/008536233 B2.

Currently, worldwide, sufficient gas reserves are presented as feed for syngas production, while the gas quality, in particular with respect to the composition, varies significantly. Concomitantly, the usability of these natural gas reserves is limited. In particular, low-calorific gases and/or gases having a high content of inert constituents, such as, e.g., CO₂, may currently only be used with difficulty, or even not at all, since processing processes of the prior art are frequently uneconomic.

The reaction of a feed gas having a high inert content to produce chemical products demands a comparatively higher feed gas amount, in such a manner that the costs for apparatuses and the energy requirement for operating apparatuses and also for cooling and heating process streams increase correspondingly.

Against this background, the object of the invention is to improve a process of the type stated at the outset.

According thereto, it is provided according to the invention that a process for generating syngas from a CO₂-rich and hydrocarbon-containing feed gas is described wherein a CO₂-rich and hydrocarbon-containing feed gas is provided and is reacted in a syngas generation step by means of partial oxidation and/or steam reforming to give an H₂- and CO-comprising syngas. The invention provides that at least CO₂ is removed from the feed gas in a scrubbing of the feed gas by means of a scrubbing medium (e.g. solvent), before the feed gas is fed to the syngas generation step. During the scrubbing, a CO₂-rich stream is generated that has a pressure in the range from 20 bar to 100 bar, and the CO₂-rich stream can then be is used as feed for a synthesis and/or to support the extraction of oil, wherein the CO₂-rich stream is injected into an oil deposit in order to increase the pressure in the oil deposit.

A CO₂-rich feed gas in the present case is taken to mean a feed gas that has a CO₂ content of at least 10% by volume, 20% by volume, 30% by volume, 40% by volume, 50 or at least 60% by volume.

In the said scrubbing, preferably the CO₂ is dissolved physically in the scrubbing medium, which can be methanol or dimethyl ether (DME). In addition, the scrubbing medium can comprise methanol and/or DME.

Preferably, therefore, in the scrubbing, a cold, methanol-containing scrubbing medium is used as physical solvent for separating off CO₂ from the feed gas stream. The feed gas stream in this case is contacted with the scrubbing medium, wherein CO₂ is physically dissolved in the scrubbing medium. Since the physical solubility of the gaseous components of the feed gas in the scrubbing medium decreases with falling temperature, the CO₂ absorption in the scrubbing medium is preferably performed at low temperatures in the range from approximately −35° C. to −65° C., and also preferably at a pressure in the range from 20 bar to 60 bar.

On account of the differing solubility of possibly further components of the feed gas (in particular sulfur compounds) in the preferably methanol-containing scrubbing medium, it is possible in said scrubbing to separate off CO₂ separately from one or more further components of the feed gas. A further component of the feed gas can be, e.g. sulfur compounds such as, for example, H₂S, CS₂, COS and/or HCN. Isolated byproducts of this type can therefore be likewise further used separately.

In particular, in the scrubbing according to the invention, the feed gas is passed into an adsorption column and brought into contact, e.g. in counterflow, with the preferably methanol-containing scrubbing medium.

On account of the different solubility coefficients of the individual components with respect to the scrubbing medium, individual components are enriched in defined regions within the absorption column. For example, the absorption column has a first section having an increased fraction of sulfur components (inter alia H₂S and COS). In addition, the absorption column has a second section having an increased fraction of CO₂. Finally, the absorption column has a third section in which substantially the feed gas which is freed from CO₂ and optionally said sulfur compounds, is present. The scrubbing medium in the absorption appliance in this case preferably has a temperature and a pressure in the abovementioned ranges.

Preferably, the feed gas is taken off from the third section and fed to the syngas generation.

The CO₂-laden scrubbing medium is preferably run from the second section of the adsorption column to a desorption column. In the desorption column, the CO₂ is removed from the scrubbing medium by means of an expansion (the solubility of the individual components falls at lower pressure) and in this case also separated off from the further acid gas components possibly present (e.g. H₂S and COS) which are still dissolved in the scrubbing medium. Alternatively, or in addition, the CO₂ can be separated off from the scrubbing medium in the desorption column by introduction of a stripping gas (e.g. N₂). The CO₂ that is separated off collects in this case in a corresponding section of the desorption column and can be taken off from there.

Preferably, the CO₂-rich stream, generated in this manner, for example, which preferably has at least a CO₂ content of 99% by volume, and which contains the CO₂ that is separated off, is provided at a pressure (dependent on the pressure of the feed gas) in the range from preferably 10 bar to 100 bar and is preferably fed to a further use.

Preferably (see also below), this CO₂-rich stream is used as feed for a synthesis, in particular a methanol synthesis, e.g. catalytically according to

CO₂+3H<->CH₃OH+H₂O.

Alternatively, or in addition, the CO₂-rich stream, according to an embodiment of the invention is used to support the extraction of oil (“Enhanced Oil Recovery” or EOR for short), wherein the CO₂-rich stream is injected into an oil deposit in order to increase the oil production rates or oil production yield, e.g. by increasing the deposit pressure. In addition, CO₂ can also be used as an additive to a flooding medium which is introduced into the oil deposit.

In the desorption column, in addition, a further section forms in which substantially (where present) said sulfur components are dissolved in the scrubbing medium.

In addition, the scrubbing medium can be passed into the desorption column from the further section of the absorption column, which further section has an elevated fraction of sulfur components, in such a manner that any CO₂ present can be removed from the scrubbing medium that is enriched with sulfur components.

The scrubbing medium from the further section of the desorption column, which scrubbing medium substantially comprises those sulfur components, is from the further section, preferably into a hot regeneration column in which removal of the sulfur components that are still present in the scrubbing medium is performed by means of heating the scrubbing medium. The resultant gas stream containing the sulfur components can then be fed to a further use.

The above-described scrubbing process using methanol as scrubbing medium is also termed rectisol process.

According to a further embodiment of the invention, it is provided that the feed gas stream that is freed in this manner from CO₂ and any further components is conducted downstream of said scrubbing through an adsorber unit, wherein one or more sulfur compounds that are still present in the feed gas are adsorbed in the adsorber unit and in this case removed from the feed gas.

Downstream of said scrubbing, the feed gas stream still preferably only has a CO₂ content of up to 1000 ppm. The abovementioned sulfur compounds, downstream of said scrubbing, preferably in each case are still only present at a content in the feed gas stream of up to 1000 ppm.

The adsorber unit downstream of the scrubber serves, in particular, to decrease further the low concentrations of the unwanted compounds still present in the feed gas, in such a manner that preferably CO₂ and possibly said sulfur compound are in each case still present with a maximum content of 10 ppm in the feed gas.

In the syngas generation step, for the syngas generation, as mentioned at the outset, partial oxidation (POX) and/or steam reformation can be used.

The feed gas stream preferably has one or more of the following components or hydrocarbons that are reacted in the syngas generation step to form the H₂- and CO-comprising syngas: CH₄, H₂O, CO₂.

In the partial oxidation, the feed gas stream that is prepurified as described above and which has, e.g. natural gas or CH₄, is substoichiometrically reacted in an exothermic process. Reaction products are primarily hydrogen and carbon monoxide which are obtained in accordance with:

CₙHₘ+n/2O₂->nCO+m/2H₂.

In the stream reformation, the feed gas stream that is prepurified as described above which has, e.g. natural gas or CH₄, is mixed with superheated process steam or steam in accordance with a steam/carbon ratio sufficient for the reformation. Then, this gas mixture is heated and distributed among the catalyst-filled reactor tubes of the furnace or reformer used. The mixture preferably flows from top to bottom through the reactor tubes that are arranged in vertical rows. On flowing through the preferably externally-fired reactor tubes, the hydrocarbon/steam mixture reacts with formation of hydrogen and carbon monoxide, e.g. in accordance with:

CnHm+nH₂O=>nCO+(n+m)/2H₂ (1)

CH₄+H₂O<=>CO+3H₂ (2)

CO+H₂O<=>CO₂+H₂ (3).

Since the heat balance for the main reactions (1)-(2) is endothermic, the required heat is fed via a combustion process in the furnace used.

According to a preferred embodiment of the invention it is provided that the syngas that is generated is divided into first and second syngas substreams, wherein the first syngas substream is used as feed for a synthesis, and wherein the second syngas substream is subjected to a watergas-shift reaction in accordance with

CO+H₂O<->CO₂+H₂,

wherein CO of the second syngas substream is reacted with H₂O to form H₂ and CO₂ in order to reduce the CO content in the second syngas substream and to increase the hydrogen content in the second syngas substream.

Preferably, the reduction of the CO₂ content in the feed gas in the scrubbing is set in dependence on a use of the syngas provided downstream of the syngas generation and/or in dependence on a desired ratio of CO to H₂ in the syngas.

According to a preferred embodiment of the invention, it is additionally provided that the second syngas substream is subjected after the watergas-shift reaction to a pressure-swing adsorption, wherein CO₂ present in the second syngas substream and also CO, H₂, CH₄ and/or H₂O additionally present therein is adsorbed to an adsorber at a first pressure and an H₂-containing stream is generated, and wherein the adsorber is regenerated at a second pressure that is lower than the first pressure, wherein adsorbed CO₂ is desorbed and wherein the adsorber is purged with an H₂-containing purge gas stream, to remove the desorbed CO₂.

Preferably, this purge gas stream is used as fuel, e.g. for providing heat in a furnace for carrying out the (above-described) steam reformation. As an alternative hereto, e.g. when using POX (see above), the purge gas can also be burnt in a different combustion appliance, e.g. to generate and/or superheat steam or process steam.

If partial oxidation (see above) is used in the syngas generation step, according to a preferred embodiment of the invention, oxygen is separated off from air (e.g. in a cryogenic air separation plant) and used as oxidizing agent in the partial oxidation, wherein the oxygen or oxidizing agent stream is added to the feed gas downstream of the scrubbing, downstream of the adsorber unit and also upstream of the syngas generation step to the feed gas. Preferably, as oxidizing agent, pure oxygen is used that only has impurities below 5% by volume.

According to a preferred embodiment of the invention, it is provided that the first syngas substream is reacted in a Fischer-Tropsch synthesis to form a crude product stream (synthetic crude oil) which comprises light hydrocarbons having four or fewer carbon atoms, heavy hydrocarbons having five or more carbon atoms, and also unreacted syngas.

In this case, it is preferably provided that a residual gas comprising light hydrocarbons and also unreacted syngas is separated off from the crude product stream or from the synthetic crude oil generated (also termed synthetic crude) and recirculated at least in part to the Fischer-Tropsch synthesis as feed, wherein some of this residual gas is recirculated as feed into the steam retformation and/or partial oxidation and/or is used as fuel.

Preferably, it is further provided that hydrogen from the H₂-containing stream, which is obtained in the pressure-swing adsorption, is used for hydrogenation of heavy hydrocarbons (e.g. aromatics) and/or oxygenated compounds of the crude product stream, and the crude product stream is divided hereinafter into one or more hydrocarbon-containing product streams.

Alternatively, or in supplementation, the syngas produced, according to a further embodiment of the invention, can also be used for a methanol synthesis.

In this case, the first syngas substream is preferably reacted in a methanol synthesis to form a methanol-comprising crude product stream, wherein preferably methanol present in the crude product stream is separated off from the unreacted syngas present in the crude product stream, generating a methanol product stream, wherein the unreacted syngas separated off is preferably recirculated as feed to the methanol synthesis.

In the case of a methanol synthesis, of course, the hydrogen obtained in the pressure-swing adsorption can also be provided as hydrogen product.

Independently of the synthesis used downstream of the syngas generation, the syngas generated in the syngas generation step is preferably cooled with water, wherein steam is generated. This is preferably used to generate electrical energy, wherein the steam is preferably superheated in advance in a furnace used in said steam reformation or in another combustion appliance.

Further features and advantages of the invention will be explained hereinafter in the description of the figures of exemplary embodiments of the invention with reference to the figures.

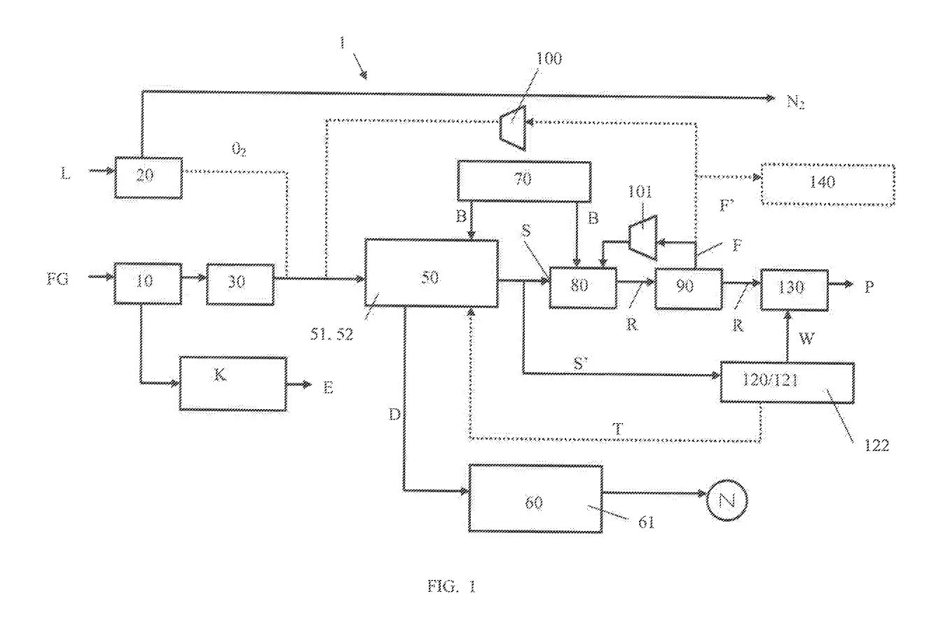

FIG. 1 shows a schematic depiction of a process according to the invention for producing a syngas from a CO₂-rich feed gas, wherein the syngas produced is used in a Fischer-Tropsch synthesis.

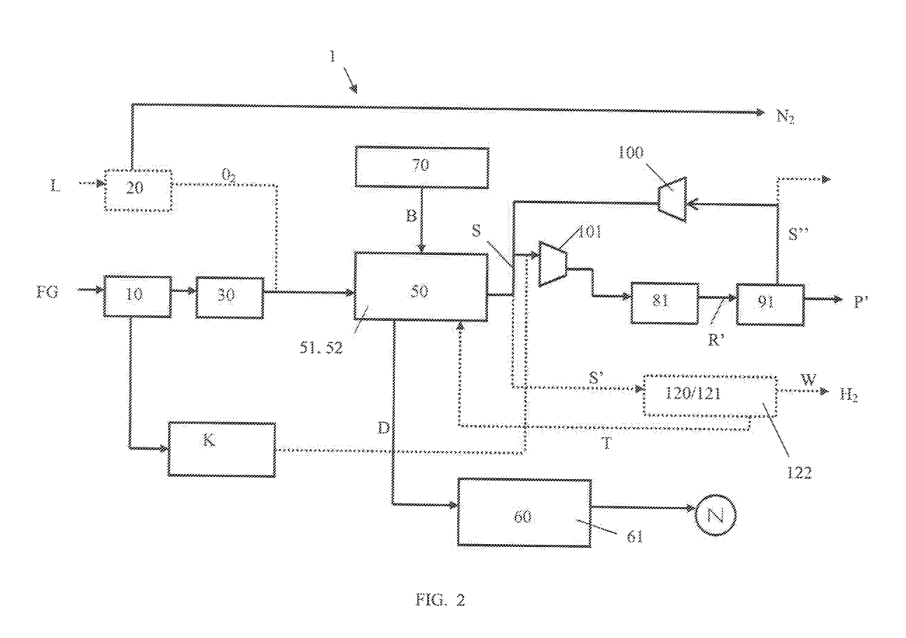

FIG. 2 shows a schematic depiction of a process according to the invention for producing a syngas from a CO₂-rich feed gas, wherein the syngas produced is used in a methanol synthesis.

FIG. 1 shows a schematic depiction of a plant 1 and of a process for generating a syngas from a CO₂-rich and hydrocarbon-rich feed gas FG, in particular natural gas, which according to an example, in addition to CO₂ at a content of 10% by volume to 70% by volume and possibly one or more sulfur compounds, such as e.g. H₂S, CS₂, COS and/or HCN, each at a content in the range of up to 5% by volume, comprises at least one of the following hydrocarbons or substances: 25% by volume to 95% by volume CH₄, 5% by volume to 75% by volume CO₂, up to 5% by volume: ethane, up to 3% by volume propane, up to 2% by volume butane, up to 3% by volume pentane, up to 5% by volume nitrogen.

The feed gas stream FG, before a reaction to form syngas (comprising H₂ and CO) is subjected according to the invention to a scrubbing in order to remove at least CO₂ and sulfur components possibly present such as e.g. H₂S, CS₂, COS from the feed gas FG. In this scrubbing 10 also designated acid gas scrubbing (in particular Rectisol process), CO₂ and any sulfur components possibly present are preferably separated off from the feed gas FG as described above, wherein preferably CO₂ and those sulfur components are separated off separately. A CO₂-rich stream or a CO₂ stream K (in particular having up to 75% by volume C)₂) is generated hereby, which has a pressure in the range from 15 bar to 100 bar.

The CO₂-rich stream K can be used, e.g. as feed for a synthesis, e.g. for a methanol synthesis 81 according to FIG. 2, or e.g. for supporting the extraction of oil, wherein the CO₂-rich stream K can be injected, e.g. into an oil deposit E in order to increase the deposit pressure.

Downstream of the acid gas scrubbing 10, the feed gas stream FG, in addition, is freed from traces of CO₂ and/or sulfur compounds still present, preferably in an adsorber unit 30, wherein the content of sulfur components is reduced to below 10 ppb, for example by means of a guard bed.

Hereafter, the feed gas stream is reacted in a syngas generation step 50 to form syngas (containing H₂ and CO). For this purpose, a partial oxidation or a steam reformation can be used.

In the steam reformation, the prepurified feed gas stream FG is mixed as described above with steam and reacted to syngas in reactor tubes, in which a suitable catalyst is arranged, at a temperature in the range from, e.g. 700° C. to 950 C and also a pressure in the range from, e.g. 20 bar to 50 bar, which syngas is then cooled and dried.

Alternatively, or supplementally, a partial oxidation can also be used, in which the feed gas FG is reacted, as described above, with oxygen at a temperature in the range from, e.g., 1100° C. to 1300° C., and a pressure in the range from, e.g., 20 bar to 100 bar, to form syngas. As oxidizing agent, preferably pure oxygen is used, which is generated by cryogenic air separation 20 and is added to the feed gas FG downstream of the acid gas scrubbing 10, downstream of the adsorber unit 30 and also upstream of the syngas generation step 50.

The syngas generated is divided into first and second syngas substreams S. S′ wherein the first syngas substream S (85 to 95% by volume) is fed as feed to a Fischer-Tropsch synthesis 80, and wherein the second syngas substream S′ (5 to 15% by volume) is subjected to a water-gas shift reaction 120 in which CO of the second syngas substream S′ is reacted with H₂O to form H₂ and CO₂ in order to reduce the CO content in the second syngas substream S′ and to increase the hydrogen content in the second syngas substream S′

After the water-gas shift reaction 120, the second syngas substream S′ is subjected to a known pressure-swing adsorption 121, wherein CO₂ present in the second syngas substream S′ is adsorbed to at least one adsorber 122 at a first pressure (e.g. in the range from 15 bar to 35 bar), and also a temperature in the range from 20° C. to 80° C. and an H₂-containing stream W (in particular having an H₂ content from 85 to 97% by volume) is generated, and wherein the adsorber 122 is regenerated at a second pressure (e.g. in the range from 20 bar to 35 bar) and also a temperature in the range from 40° C. to 120° C., wherein adsorbed CO₂ is desorbed and wherein the adsorber 122 is purged with H₂, generating an H₂-containing purge gas stream T, to remove the desorbed CO₂ (and also any other desorbed components). Preferably, a plurality of, in particular, two or four, adsorbers, are used in the pressure-swing adsorption, in order that as far as possible one adsorber can always be operated in the adsorption mode in such a manner that hydrogen can be delivered semicontinuously. The purge gas stream T can, e.g., be burnt as fuel in a furnace 51 to carry out the steam reformation and/or can be used as fuel to generate and/or superheat steam.

In the said Fischer-Tropsch synthesis 80, the first syngas substream S is reacted in a known manner to form a crude product stream R which comprises light hydrocarbons having four or fewer carbon atoms, heavy hydrocarbons having five or more carbon atoms, and also unreacted syngas. Water B required for the synthesis 80 is provided by means of a water supply 70. From the crude product stream R, a residual gas F comprising the light hydrocarbons and also unreacted syngas is separated off, wherein at least a part of the residual gas F, after compression in a compressor 101 (e.g. to a pressure in the range from 15 bar to 35 bar), is recirculated as feed to the Fischer-Tropsch synthesis 80. A further part F′ of the residual gas F can, after compression in a compressor 100 (e.g. to a pressure in the range from 20 bar to 50 bar), be recirculated as feed into the steam reformation 50 and/or be used as fuel 140.

The H₂-containing stream W generated in the pressure-swing adsorption 121, in addition, is used e.g. for hydrogenation (130) of heavy hydrocarbons of the crude product stream R of the Fischer-Tropsch synthesis. The treated crude product stream R is divided into one or more hydrocarbon-containing product streams P that can have different hydrocarbon fractions.

FIG. 2 shows a further exemplary embodiment of the invention, in which, in contrast to FIG. 1, a Fischer-Tropsch synthesis is not carried out, but rather a methanol synthesis 81. In this case, the first syngas substream S is compressed in a compressor 101 (e.g. to a pressure in the range from 20 bar to 100 bar) and reacted in the methanol synthesis 81 to form a methanol-comprising crude product stream R′, wherein methanol present in the crude product stream R′ is separated 91 from the unreacted syngas S″ present in the crude product stream R′, wherein a methanol product stream P′ is generated. The unreacted syngas S″ separated off is compressed in a compressor 100 (e.g. to a pressure in the range from 40 bar to 100 bar) and recirculated as feed to the methanol synthesis 81, more precisely via the other compressor 101 (wherein, in particular, a pressure elevation to a pressure in the range from 40 bar to 100 bar proceeds). The H₂-containing stream W obtained in the pressure-swing adsorption 121 can be provided, e.g. as hydrogen product.

In addition, in the embodiment according to FIG. 2, the CO₂-rich stream K can also be used as feed for the methanol synthesis 81 (cf. FIG. 1), wherein the stream K is conducted to the methanol synthesis via the compressor 101 and in this case is compressed to a pressure in the range from 40 bar to 100 bar.

In the embodiments according to FIGS. 1 and 2, in each case the syngas generated in the syngas generation step 50 is cooled with water B of the water supply 70, wherein steam D is generated that can be used to generate electrical energy 60. In this case, the steam D can be superheated, e.g. in the furnace 51 of the steam reformation 50, or in another combustion furnace 52, and then used for generating electrical energy, e.g. in a steam turbine 61.

Ultimately, the teaching according to the invention permits a comparatively low inert content or CO₂ content to be obtained in the syngas stream, wherein the plant can overall be made smaller, manages with a lower energy consumption and the process streams that are to be recirculated are advantageously comparatively smaller. In the POX, a lower oxygen consumption becomes possible.

### LIST OF REFERENCE SIGNS

| | |
|---|---|
| 1 | Plant for syngas production and also synthesis of |
| | hydrocarbons |
| 10 | Scrubbing for CO₂ removal |
| 20 | Air separation unit |
| 30 | Adsorber unit for desulfurization |
| 50 | Syngas generation step and also syngas cooling |
| 51 | Furnace for steam reformation |
| 52 | Combustion furnace |
| 60 | Energy generation |
| 61 | Steam turbine |
| 70 | Water supply |
| 80 | Fischer-Tropsch synthesis |
| 81 | Methanol synthesis |
| 90, 91 | Separation |
| 100, 101 | Compressor |
| 120 | Water-gas Shift reaction |
| 121 | Pressure-swing adsorption |
| 130 | Product workup |
| 140 | Fuel system or fuel supply |
| B | Water |
| E | Oil deposit |
| F, F′ | Residual stream |
| K | CO₂-rich stream |
| L | Air |
| FG | Feed gas |
| P, P′ | Product stream |
| R, R′ | Crude product stream |
| S | First syngas substream |
| S′ | Second syngas substream |
| S″ | Unreacted syngas |
| T | Purge gas |
| W | Hydrogen-containing stream |

1. Process for generating a syngas from a CO2-rich and hydrocarbon-containing feed gas, wherein a CO2-rich and hydrocarbon-containing feed gas is provided and is reacted in a syngas generation step by means of partial oxidation or steam reforming to give an H2- and CO-comprising syngas,
characterized in that at least CO2 is removed from the feed gas in a scrubbing of the feed gas by means of a scrubbing medium, before the feed gas is fed to the syngas generation step, wherein, during the scrubbing, a CO2-rich stream is generated that has a pressure in the range from 20 bar to 100 bar, and wherein the CO2-rich stream is used as feed for a synthesis or to support the extraction of oil, wherein the CO2-rich stream is injected into an oil deposit in order to increase the pressure in the oil deposit.

2. Process according claim 1, characterized in that the feed gas is conducted downstream of the scrubbing through an adsorber unit, wherein one or more sulfur compounds that are still present in the feed gas are adsorbed in the adsorber unit and in this case removed from the feed gas.

3. Process according to claim 1, characterized in that the syngas that is generated is divided into first and second syngas substreams, wherein the first syngas substream is used as feed for a synthesis, and wherein the second syngas substream is subjected to a water-gas shift reaction, wherein CO of the second syngas substream is reacted with H2O to form H2 and CO2 in order to reduce the CO content in the second syngas substream and to increase the hydrogen content in the second syngas substream.

4. Process according to claim 1, characterized in that the reduction of the CO2 content in the feed gas in the scrubbing is set in dependence on a use of the syngas provided downstream of the syngas generation or in dependence on a desired ratio of CO to H2 in the syngas.

5. Process according to claim 3, characterized in that the second syngas substream is subjected after the water-gas shift reaction to a pressure-swing adsorption, wherein CO2 present in the second syngas substream is adsorbed to an adsorber at a first pressure and an H2-containing stream is generated, and wherein the adsorber is regenerated at a second pressure that is lower than the first pressure, wherein adsorbed CO2 is desorbed and wherein the adsorber is purged with H2, generating an H2-containing purge gas stream, to remove the desorbed CO2.

6. Process according to claim 5, characterized in that the purge gas stream is used as fuel, wherein the purge gas stream is burnt in a furnace to carry out the steam reformation or wherein the purge gas stream is burnt in a combustion furnace to generate or superheat steam.

7. Process according to claim 2, characterized in that oxygen is separated off cryogenically from air and used as oxidizing agent in the partial oxidation, wherein the oxygen is added to the feed gas downstream of the scrubbing, downstream of the adsorber unit and also upstream of the syngas generation step to the feed gas.

8. Process according to claim 5, characterized in that the synthesis is a Fischer-Tropsch synthesis, wherein the first syngas substream is reacted in the Fischer-Tropsch synthesis to form a crude product stream which comprises light hydrocarbons having four or fewer carbon atoms, heavy hydrocarbons having five or more carbon atoms, and also unreacted syngas.

9. Process according to claim 8, characterized in that a residual gas comprising light hydrocarbons and also unreacted syngas is separated off from the crude product stream and recirculated at least in part to the Fischer-Tropsch synthesis as feed, wherein some of the residual gas is recirculated as feed into the steam reformation or partial oxidation or is used as fuel.

10. Process according to claim 9, characterized in that hydrogen from the H2-containing stream is used for hydrogenation of heavy hydrocarbons of the crude product stream, wherein the crude product stream is divided hereinafter into one or more hydrocarbon-containing product streams.

11. Process according to claim 3, characterized in that the synthesis is a methanol synthesis, wherein the first syngas substream is reacted in the methanol synthesis to form a methanol-comprising crude product stream.

12. Process according to claim 11, characterized in that methanol present in the methanol-comprising crude product stream is separated from unreacted syngas present in the methanol-comprising crude product stream, generating a methanol product stream, wherein the unreacted syngas separated off is recirculated as feed to the methanol synthesis.

13. Process according to claim 1, characterized in that the syngas generated in the syngas generation step is cooled with water, wherein steam is generated that is used to generate electrical energy, wherein the steam is superheated in a furnace for carrying out the steam reformation or in another combustion furnace, and is then used in a steam turbine to generate electrical energy.
